(19) United States
(12) Patent Application Publication
(10) Pub. No.: US 2023/0281890 A1
(43) Pub. Date: Sep. 7, 2023

(54) COMPUTER-IMPLEMENTED METHOD FOR DETERMINING NUCLEAR MEDICAL IMAGE DATA SETS, IMAGING DEVICE AND ELECTRONICALLY READABLE STORAGE MEDIUM

(71) Applicant: Siemens Healthcare GmbH, Erlangen (DE)

(72) Inventors: Matthias FENCHEL, Erlangen (DE); Hans WEHRL, Erlangen (DE); Bernard BENDRIEM, Knoxville, TN (US)

(73) Assignee: Siemens Healthcare GmbH, Erlangen (DE)

(21) Appl. No.: 18/174,773

(22) Filed: Feb. 27, 2023

(30) Foreign Application Priority Data
Mar. 1, 2022 (EP) 22159399.9

(51) Int. Cl. G06T 11/00; A61B 5/055; A61B 5/00

(52) U.S. Cl. CPC G06T 11/005; A61B 5/055; A61B 5/0035; G06T 2211/424; G06T 2210/41

(57) ABSTRACT

A computer-implemented method for determining at least one nuclear medical image data set in nuclear medical imaging using an imaging device includes acquiring nuclear medical raw data sets of a region of interest of a patient in respective acquisition steps during a progression of a tracer in the region of interest; reconstructing at least one nuclear medical image data set from the nuclear medical raw data sets for each acquisition step; determining motion data describing a motion of the patient between acquisition steps from source data describing the motion in the region of interest; and applying motion correction to at least one of the nuclear medical raw data sets or the at least one nuclear medical image data set based on the motion data.

### CROSS-REFERENCE TO RELATED APPLICATION(S)

The present application claims priority under 35 U.S.C. § 119 to European Patent Application No. 22159399.9, filed Mar. 1, 2022, the entire contents of which are incorporated herein by reference.

### FIELD

One or more example embodiments of the present invention concerns a computer-implemented method for determining at least one nuclear medical image data set in nuclear medical imaging using an imaging device. One or more example embodiments of the present invention further concerns an imaging device, a computer program and an electronically readable storage medium.

### RELATED ART

Nuclear medical imaging, which may also be called emission tomography, is an imaging technique in which radioactively labeled substances, so-called tracers, inside the body of a patient are detected. Examples for nuclear medical imaging comprise positron emission tomography (PET) and single photon emission computer tomography (SPECT). From the resulting nuclear medical raw data sets, for example sinogram data and/or list-mode data, which describe radiation measuring events, nuclear medical image data sets showing the distribution of the tracer can be reconstructed. In basic reconstruction approaches, backprojection, in particular filtered backprojection (FBP), may be employed. However, such images are often noisy, so that advanced, iterative reconstruction methods have been proposed, in particular MLEM (maximum likelihood expectation maximization) and/or OSEM (ordered subsets expectation maximization).

In some nuclear medical imaging applications, nuclear medical raw data of the same anatomical region are acquired in different acquisition steps, in particular separated in time. For example, in dynamic nuclear medical imaging, a time period of progression of a tracer inside the patient may be divided into multiple frames as acquisition steps, wherein a nuclear medical image data set for each frame can be reconstructed from the nuclear medical raw data acquired in this frame. For reasons of clearer distinction between states of tracer distribution, a time interval may be provided between the acquisition times of different frames.

Furthermore, combined-modality workflows, for example PET-magnetic resonance (MR) workflows, have been proposed, in which nuclear medical raw data from an anatomical region is acquired in two different acquisition steps separated in time, in particular if regions of interest, which are larger than the field of view of the imaging device, are examined. In this case, multiple predefined positions of the field of view, such that the whole region of interest is covered, may be used for acquisition, in particular multiple predefined patient table positions. In particular, whole-body scans may be executed using imaging devices providing an additional imaging modality additional to the nuclear medical imaging modality, in particular PET-MR devices.

Here, it has been proposed to change the nuclear medical raw data assigned to different patient table positions with respect to the data volume in such a way that the recording times corresponding to the change to data volumes are matched to each other at different table positions, as described, for example, in U.S. Pat. No. 8,781,195 B2. It was proposed to increase the recorded nuclear medical raw data assigned to a table position by further acquisition at this table position in another acquisition step.

In those cases, in other words, each anatomical region is imaged several times during an examination. However, a reading physician may want to obtain a single PET image with all summed counts of the individual passes for diagnostic purposes. Since the acquisition steps result in acquisition at different time points, changes may occur, in particular patient motion between different measurements of nuclear medical raw data sets of different acquisition steps. Furthermore, attenuation maps for attenuation correction are usually acquired at the beginning of the examination, in particular in a first acquisition step, and may not properly represent the motion state of each acquisition step. This could lead to nuclear medical image quality problems, for example blurring effects and/or quantification biases, for example in a final summed full-count image.

### SUMMARY

While, in the state of the art, it has already been proposed to provide motion correction regarding periodical physiological motions like respiration and/or cardiac motion within an acquisition step, for example using techniques like “BodyCompass”, “OncoFreeze” or “CardiacFreeze”, these approaches cannot account for image quality problems between acquisition steps.

One or more example embodiments of the present invention provides a method for increasing the image quality and spatial correspondence of nuclear medical image data sets based on raw data acquired in different acquisition periods, in particular spaced in time.

According to one or more example embodiments, a computer-implemented method for determining at least one nuclear medical image data set in nuclear medical imaging using an imaging device includes acquiring nuclear medical raw data sets of a region of interest of a patient in respective acquisition steps during a progression of a tracer in the region of interest; reconstructing at least one nuclear medical image data set from the nuclear medical raw data sets for each acquisition step; determining motion data describing a motion of the patient between acquisition steps from source data describing the motion in the region of interest; and applying motion correction to at least one of the nuclear medical raw data sets or the at least one nuclear medical image data set based on the motion data.

According to one or more example embodiments, the region of interest is larger than a field of view of the imaging device, the acquiring includes at least one of sweeping, during each acquisition step, the field of view a whole region of interest using at least one of multiple predefined positions of the field of view or a continuous movement of the field of view, or at least one of (i) determining, for all acquisition steps, a series of nuclear medical image data sets or (ii) the region of interest comprises a whole body of the patient.

According to one or more example embodiments, the determining includes at least one of, registering the source data relating to one of the acquisition steps to source data from another acquisition step to determine motion data between the acquisition steps, or using optical flow algorithms to determine the motion data.

According to one or more example embodiments, the determining further includes at least one of, registering neighboring acquisition steps in succession during the acquiring, from a first acquisition step or a last acquisition step, or using preliminary reconstructed images from the nuclear medical raw data sets as source data to register.

According to one or more example embodiments, the applying includes applying the motion correction to a reconstruction result in an image space, applying the motion correction in a raw data space during iterative reconstruction, or the nuclear medical raw data sets are PET raw data sets, wherein lines of response are displaced according to the motion data for motion correction.

According to one or more example embodiments, the method further includes applying attenuation correction based on an attenuation map to the at least one nuclear medical image data set, and the applying the motion correction applies the motion correction to the attenuation map based on the motion data.

According to one or more example embodiments, the source data is at least partly acquired by an acquisition device of an additional modality different from the nuclear medical imaging during the acquisition steps, the acquisition device is registered to the imaging device.

According to one or more example embodiments, the acquisition device is at least one of, at least one of a CT device or an MRI device integrated into the imaging device, a camera, a radar device, or an ultrasound device.

According to one or more example embodiments, the method further includes applying attenuation correction based on an attenuation map, the attenuation map being determined from attenuation correction MRI data acquired by the acquisition device during at least one of the acquisition steps, the attenuation correction MRI data being used as at least a part of the source data.

According to one or more example embodiments, the acquiring includes acquiring acquisition correction MRI data in each acquisition step to determine a respective attenuation map for the acquisition step, or acquiring acquisition correction MRI data only in one acquisition step, wherein, for the other acquisition steps, MRI data are acquired and at least partly used as source data.

According to one or more example embodiments, the source data is from different modalities.

According to one or more example embodiments, the determining the motion data determines motion data time-resolved within the acquisition steps.

According to one or more example embodiments, an imaging device for determining at least one nuclear medical image data set in nuclear medical imaging includes a control device including, an acquisition unit configured to acquire nuclear medical raw data sets of a region of interest of a patient in respective acquisition steps during a progression of a tracer in the region of interest, a reconstruction unit configured to reconstruct the at least one nuclear medical image data set from the nuclear medical raw data sets for each acquisition step, and a motion correction unit configured to determine motion data describing a motion of the patient between acquisition steps from source data describing the motion in the region of interest and apply motion correction to at least one of the nuclear medical raw data sets or the at least one nuclear medical image data set based on the motion data.

According to one or more example embodiments, a non-transitory electronically readable storage medium includes instructions that, when executed by a control device of an imaging device, causes the imaging device to perform a method according to one or more example embodiments.

### BRIEF DESCRIPTION OF THE DRAWINGS

Objects and features of one or more example embodiments of the present invention will become apparent from the following detailed description considered in conjunction with the accompanying drawings. The drawings, however, are only principle sketches designed solely for the purpose of illustration and do not limit the invention. The drawings show:

FIG. 1 a general flowchart of methods according to one or more example embodiments of the invention,

FIG. 2 a first acquisition schedule according to one or more example embodiments,

FIG. 3 a second acquisition schedule according to one or more example embodiments,

FIG. 4 a flowchart regarding an embodiment for determining motion data, and

FIG. 5 an imaging device according to one or more example embodiments of the invention.

### DETAILED DESCRIPTION

In a computer-implemented method for determining at least one nuclear medical image data set in nuclear medical imaging using an imaging device, according to one or more example embodiments of the present invention,
multiple nuclear medical raw data sets of a region of interest of a patient are acquired in respective acquisition steps during the progression of a tracer in the region of interest,
at least one nuclear medical image data set is reconstructed from the nuclear medical raw data sets of the acquisition steps,
motion data describing the motion of the patient between acquisition steps is determined from source data describing the motion in the region of interest and motion correction is applied to the series of nuclear medical raw data sets and/or the at least one nuclear medical image data set according to the motion data.

Hence, the at least one nuclear medical image data set relates to a certain motion state. It is proposed to use motion information from different acquisition steps to provide nuclear medical image data sets of improved quality and/or spatial correspondence. This is in particular preferable if there is a time interval between the acquisition of the same sub-region of the region of interest, for example a certain position of the field of view with respect to the patient. While the method described here is also applicable to dynamic nuclear medical imaging, where the region of interest may be equal to or fully comprised by the field of view, particularly preferred embodiments relate to cases where, due to the field of view being smaller than the region of interest, a certain time interval passes between acquisitions of the same sub-region of the region of interest, for example defined by a certain patient table position.

Hence, in preferred embodiments, the region of interest may be larger than the field of view of the imaging device, wherein, during each acquisition step, the field of view sweeps the whole region of interest by using multiple predefined positions of the field of view and/or continuous movement of the field of view with respect to the patient and/or wherein a series of nuclear medical image data sets is determined for all acquisition steps. In this particularly preferred embodiment, dynamic nuclear medical imaging can also be implemented for cases in which the field of view is smaller than the region of interest by repeatedly sweeping through the region of interest during the multiple acquisition steps, hence measuring nuclear medical raw data from different distribution states over the progression of the tracer in the region of interest. It is noted that these multiple passes of each sub-region can also be triggered or required by workflows as described in U.S. Pat. No. 8,781,195 B2, but may advantageously be dedicatedly planned to allow dynamic nuclear medical imaging. Having, in particular complete, nuclear medical raw data sets for each acquisition step allows to reconstruct nuclear medical image data sets for each of these acquisition steps, such that a series of nuclear medical image data sets results, describing the evolution of the tracer in a large region of the body. Generally and preferably, the region of interest may comprise the whole body of a patient (whole-body examination head to thigh or full body head to toe). To determine a full-count image data set, the single nuclear medical image sets of the acquisition steps may be summed up. Summation may happen both in image and in raw data space. In this manner, a full-count nuclear medical image data set as well as information about the change of tracer distribution in time is provided.

According to one or more example embodiments of the present invention, the result is of a particular high quality despite possible motion in the region of interest, since motion data is determined and used for motion correction, such that nuclear medical image data sets from different acquisition steps can readily be compared and/or reconstructions can be combined.

It should be noted at this point that, preferably, the acquisition times for all acquisition steps are the same for the whole region of interest; however, in some cases, different acquisition times may be necessary or advantageous, such that, to enable comparison, further image processing and/or selection of nuclear medical raw data may be necessary.

As already mentioned, usually, different predefined positions of the field of view defining sub-regions of the region of interest may be selected by using different predefined patient table positions of a patient table on which the patient is placed for examination in the imaging device. For example, nuclear medical raw data of a nuclear medical raw data set of an acquisition step may be acquired for 0.5 to 2 minutes at each of, for example, two to seven predefined positions of the field of view, in particular the patient table. It has also been proposed to acquire nuclear medical imaging raw data over the whole region of interest by continuously moving the field of view, in particular the patient table, where one or more example embodiments of the present invention can, of course, also be employed.

Generally, the motion data may comprise vector fields for elastic motion and/or rigid body transformations for rigid body motion, in particular depending on anatomical features in the field of view. For example, rigid body transformations may be employed for the head of a patient. Combinations of vector fields for elastic motion and rigid body transformations are also conceivable, in particular for different anatomical features. For example, in the head area, rigid body transformations may be determined assuming the head as being a rigid body, while in the breast and/or abdomen area, elastic motion may be assumed such that vector fields best describe this elastic motion.

In preferred embodiments, the source data relating to one of the acquisition steps may be registered to source data from another acquisition step to determine motion data between the acquisition steps and/or optical flow algorithms may be used to determine the motion data. Hence, generally known motion estimation and/or detecting algorithms can also be used in the current invention, for example image-based registration algorithms and/or optical flow algorithms. As registration and/or optical flow are preferably evaluated in source data of the same modality, resource-saving implementations of the motion estimation and/or detection algorithms can be employed.

It should be noted already at this point that it is possible to use the nuclear medical imaging raw data, in particular images derived therefrom, as source data and/or to use source data from other modalities which are acquired anyways during the acquisition. For example, if the imaging device is a combined PET-MR device, in most of the examinations, magnetic resonance imaging data (MRI data) are usually acquired in parallel to the nuclear medical raw data sets. Hence, in such a case the current invention describes a straightforward extension of any multi-pass workflow in order to improve image quality and/or evaluability. Since the required source data is available anyways, the method can work without additional acquisitions or scans.

Preferably, if nuclear medical imaging data is to be used as source data, preliminary reconstructed images from the nuclear medical raw data sets, in particular backprojected images or other images determined by fast preliminary reconstruction, may be used as source data to register. Such preliminary reconstructed images, which are not yet motion-corrected, may hence serve as a basis for registering. Here, the image quality of simply backprojected images, for example by using filtered backprojection, or other fast preliminary reconstructed images already suffices to provide the necessary degree of precision in the motion data. Hence, readily available preliminary reconstructed images may be used without further effort and/or scans.

If a registration algorithm is used for motion estimation and/or detection, in preferred embodiments, neighboring acquisition steps may be registered in succession over the acquisition interval, in particular starting from the first or last acquisition step. If acquisition steps adjacent in time are registered, the least difference in motion state is expected, increasing robustness and reliability of the determination of the motion data. For example, source data from the first acquisition step may first be registered with source data from the second acquisition step, whereafter source data from the second acquisition step is registered to source data of the third acquisition step, and so on.

For applying the motion correction, multiple approaches are contemplated, wherein application of motion correction to the nuclear medical raw data sets and/or during reconstruction is preferred. It is, however, also conceivable to apply motion correction to an, in particular preliminary, reconstruction result in the image space. For example, image transformations may be applied before summation and/or comparison to reconstruction results of acquisition steps.

In preferred embodiments, motion correction is applied in raw data space during iterative reconstruction, in particular during motion-sensitive OSEM reconstruction and/or by warping an intermediate image result according to the motion data before forward projection and unwarping of correction terms after backprojection according to the motion data. Iterative reconstruction approaches are well-known in the state of the art and most often used in expectation maximization (EM) approaches, wherein an intermediate guess for the result image is forward projected to compare the nuclear medical raw data to the forward projected intermediate image data. Depending on this comparison, correction terms are applied to yield a new intermediate image as a guess for the real tracer distribution. Hence, if motion correction is to be applied during reconstruction while still being able to compare with the acquired nuclear medical raw data, motion correction is applied as described by warping the intermediate image result according to the motion data and then respectively unwarping correction terms. Motion-sensitive iterative reconstruction approaches have already been proposed, for example as motion-sensitive ordered subset expectation maximization (OSEM).

In other preferred embodiments, the nuclear medical raw data sets may be PET raw data sets, wherein, for motion correction, the lines of response are displaced according to the motion data. While the motion correction can, as described with respect to motion-sensitive OSEM above, be applied in sinogram space, it is also possible to apply motion correction in listmode space by reordering the lines of response of the events according to the motion data.

As already discussed, motion-corrected nuclear medical image data sets may be determined for multiple acquisition steps, in particular all acquisition steps. The motion data may be used to provide voxel-wise correspondence between nuclear medical image data sets for different acquisition steps. In this case, a series of dynamic nuclear medical images results, which are comparable and can be evaluated together. Alternatively or preferably, additionally, a motion-corrected nuclear medical image data set for multiple acquisition steps, in particular all acquisition steps, may be determined as a multi-count image data set, in particular a full count image data set. This can, as discussed, preferably be realized by summing motion-corrected nuclear medical image data sets for the different acquisition steps and/or by applying motion correction during reconstruction and/or already on the nuclear medical raw data sets.

In a preferred embodiment, attenuation correction (AC) based on an attenuation map may be applied to the at least one nuclear medical image data set, wherein the attenuation map is also motion-corrected according the motion data. Attenuation correction for nuclear medical imaging is well-known and can, preferably, also be applied here. In particular in cases where an attenuation map is only determined for one of the acquisition steps, the motion data may, of course, also be used to correct the attenuation map for the other acquisition steps, further improving image quality and spatial accuracy of correspondence.

As already mentioned, at least a part of the source data for each acquisition step may be provided by nuclear medical raw data and/or images derived therefrom. In embodiments, source data may additionally or alternatively be acquired by an acquisition device of an additional modality different from the nuclear medical imaging during the acquisition steps, wherein the acquisition device is registered to the imaging device. Hence, additional data, in particular data acquired anyways during the examination, can be used to provide robust, reliable and high-quality motion data and hence motion correction.

In especially preferred embodiments, the acquisition device may be an MRI device integrated into the imaging device, which may preferably be a PET device. That is, the imaging device may be a combined PET-MR device, as proposed in the state of the art. Here, two modalities, namely magnetic resonance imaging (MRI) and positron emission tomography (PET) are provided and, by integration, already registered to each other. Such combined imaging devices are often used to derive attenuation maps from the additional modality, here MRI, as, for example, described in an article by Daniel H. Paulus et al., “Whole-Body PET/MR Imaging: Quantitative Evaluation of a Novel Model-Based MR Attenuation Correction Method Including Bone”, J Nucl Med. 56 (2015), pages 1061-1066. Such a combined device is also mainly discussed in U.S. Pat. No. 8,781,195 B2. It is noted that also computer tomography (CT) may be used as additional modality, however, this is less preferred, since a high radiation dose would be applied to the patient when CT is excessively used.

However, also other additional modalities may be employed. For example, the acquisition device can also be a camera, in particular a 3D and/or terahertz camera, and/or a radar device and/or an ultrasound device. Such acquisition devices have already been proposed as a source of motion information.

In the context of MRI as additional modality, if attenuation correction is applied using the attenuation map, preferably, the attenuation map may be determined from attenuation correction MRI data acquired by the acquisition device during at least one of the acquisition steps, in particular at least the first acquisition step, wherein the attenuation correction MRI data is used as at least a part of the source data. In particular, acquisition correction MRI data may be acquired in each acquisition step to determine a respective attenuation map for the acquisition step. However, if acquisition correction MRI data are only acquired in one acquisition step, for the other acquisition steps, further, in particular diagnostic, MRI data may be acquired and at least partly used as source data. In both cases, MRI data acquired anyways in the workflow can additionally be used as source data for determining the motion data, allowing to avoid additional scans and/or acquisition devices for the source data. As already discussed in the art, for example in the article by Daniel H. Paulus cited above, dedicated attenuation correction sequences, in particular for Dixon techniques, may be used to acquire the attenuation correction MRI data, which may also be called AC MRI data. However, further MRI data acquired using other imaging sequences may alternatively or additionally be used as source data. It is noted that, if, during an acquisition step, no AC MRI data or further MRI data is anyways acquired, additional MRI data acquisition may, of course, be provided.

In preferred embodiments, source data from different modalities may be used to determine the motion data, in particular from modalities showing different anatomical features. For example, PET data and MRI data may both be used as source data. For example, if lesions visible in PET are not visible in MRI and/or anatomical structures visible in MRI are not visible in PET, by using source data from both modalities, motion information from different anatomical features can be combined to improve the quality of the motion data.

It is noted that motion data may also be determined time-resolved within the acquisition steps and used for motion correction of data acquired within the acquisition steps, as in principle known from the state of the art, to further improve image quality and evaluability.

One or more example embodiments of the present invention further concerns an imaging device for determining at least one nuclear medical image data set in nuclear medical imaging, comprising a control device having
an acquisition unit for acquiring multiple nuclear medical raw data sets of a region of interest of a patient in respective acquisition steps during the progression of a tracer in the region of interest,
a reconstruction unit for reconstructing the at least one nuclear medical image data set from the nuclear medical raw data sets for each acquisition step, and
a correction unit for determining motion data describing the motion of the patient between acquisition steps from source data describing the motion in the region of interest and applying motion correction to the series of nuclear medical raw data sets and/or the at least one nuclear medical image data set according to the motion data.

All comments and remarks regarding the computer-implemented method according to one or more example embodiments of the present invention analogously apply to the imaging device according to one or more example embodiments of the present invention, such that the same advantages can be achieved. In particular, the control device of the imaging device is configured to perform a method according to one or more example embodiments of the present invention. The imaging device may include acquisition components controlled by the acquisition unit to acquire data. The control device may, in particular, comprise at least one processor and/or at least one storage device. Further functional units may be provided to realize preferred embodiments, in particular those described in the dependent claims.

Preferably, the imaging device is a combined imaging device (often also called hybrid imaging device), in particular a PET-MR device.

A computer program according to one or more example embodiments of the present invention can be directly loaded into the storage device of a control device of an imaging device and enables the control device to perform the steps of a method according to one or more example embodiments of the present invention when the computer program is executed on the control device. The computer program may be stored on an electronically readable storage medium according to one or more example embodiments of the present invention, which thus comprises control information comprising a computer program according to one or more example embodiments of the present invention, such that, when the electronically readable storage medium is used in a control device of an imaging device, the control device is configured to perform the steps of a method according to one or more example embodiments of the present invention. The electronically readable storage medium may preferably be a non-transitory medium, for example a CDROM.

In the following, embodiments of the current invention are discussed, wherein the nuclear medical imaging modality is positron emission tomography (PET) and a combined (or hybrid) imaging device is used, wherein the additional modality is magnetic resonance imaging (MRI). However, one or more example embodiments of the present invention is also applicable to other modalities and modality combinations, for example single photon emission computer tomography (SPECT). Other additional modalities like cameras or radar sensors may also be employed to provide source data for motion correction. It is, in particular, noted that one or more example embodiments of the present invention can also be applied to single nuclear medical imaging modalities, in particular PET, where the single nuclear imaging modality itself provides source data. Using a PET-MR device the imaging device is, however, preferred.

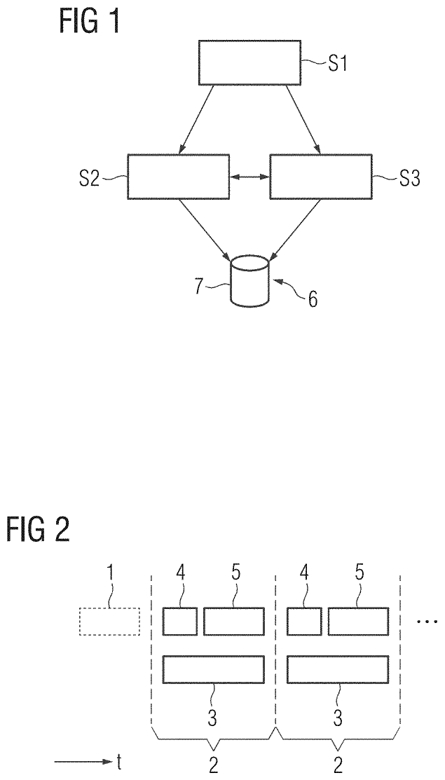

FIG. 1 shows a general flowchart for embodiments of the method according to the invention. Here, in a step S1, data are acquired by the imaging device. In the embodiments discussed here, a region of interest of a patient is to be examined, which is larger than the field of view of the nuclear medical imaging device, here PET device, of the imaging device. Hence, multiple table positions of the patient table of the imaging device (often also called bed positions) are predefined such that all subregions of the region of interest can be imaged. In particular, the examination to be performed may be a whole-body examination. For example, two to seven predefined patient table positions, which correspond to field of view positions with respect to the patient, may be used.

Nuclear medical raw data, in this case PET raw data, of the region of interest are not acquired in one pass or sweep of the region of interest using the predefined patient table positions, but in multiple acquisition steps, in particular multiple full sweeps of the region of interest, which may also be called multiple passes of the region of interest. For each of the acquisition steps, a nuclear medical raw data set, in particular a list-mode data set, is acquired. Here, in particular since multiple predefined positions of the field of view, in this case patient table positions, are successively used, there is always a time interval between the acquisition of nuclear medical raw data from the same sub-region. Of course, there may also be a pause between PET acquisitions of the different steps. For example, five predefined table positions may be used, where nuclear medical raw data are acquired for one minute in each acquisition step (pass). Hence, it will take at least four minutes until nuclear medical raw data will be acquired from the same subregion of the region of interest again. It should be noted at this point that the principles of one or more example embodiments of the present invention, as discussed here, can of course also be applied to cases where the patient table is continuously moved to sweep the region of interest.

Since the nuclear medical raw data sets of different acquisition steps are acquired at different points in time in step S1, motion effects, in particular regarding patient motion, may become relevant.

In preferred embodiments, the acquisition schedule is chosen such that dynamic nuclear medical imaging can be performed. In particular, each nuclear medical raw data set may be acquired in the same manner, that is, measurement is performed for the same acquisition times for each patient table position. In this manner, as will be further discussed below, nuclear medical image data sets may be determined for each acquisition step, providing a series of nuclear medical images describing the progression of the tracer, which was given to the patient long before the acquisition in step S1, in the region of interest.

An acquisition schedule is shown exemplarily in FIG. 2. Here, the upper blocks relate to MRI acquisitions, while the lower blocks relate to PET acquisitions. First, as illustrated by block 1, MR localizer data are acquired, such that the combined PET-MRI acquisition can be planned. After planning, in a first acquisition step 2, PET raw data of a first PET raw data set (as nuclear medical data set) are acquired for all predefined patient table positions. In parallel, since the combined imaging device allows synchronous acquisition, MRI data are acquired, namely, according to block 4, attenuation correction MRI data and, according to block 5, further MRI data, in this case diagnostic MRI data.

In the example of FIG. 2, this acquisition step 2 is repeated for a predefined number of times, for example three to six times, to, in particular, acquire, for each acquisition step 2, a PET raw data set using the same acquisition parameters.

It is noted that, in some cases, attenuation correction MRI data (AC MRI data) may only be acquired in one of the acquisition steps 2, however, acquisition in each acquisition step 2 is preferred. Furthermore, of course, the acquisition time for MRI data may, in this first example, also be shorter than the acquisition time for the PET data. Generally, attenuation correction MRI data is used to determine attenuation correction maps to apply attenuation correction to nuclear medical imaging raw and/or image data, in particular during reconstruction.

However, one or more example embodiments of the present invention can also be applied to other acquisition schedules, in which multiple PET raw data sets, optionally using different acquisition parameters, are acquired in different acquisition steps 2.

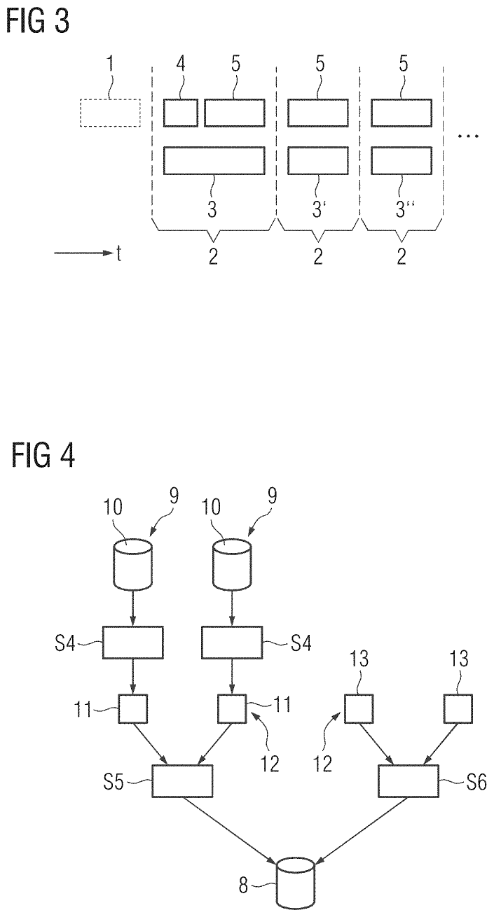

FIG. 3 shows an example where, again after the acquisition of localizer MRI data in block 1 and respective planning, in the first acquisition step 2, attenuation correction MRI data are acquired in block 4 and diagnostic MRI data are acquired in block 5. PET raw data of a full sweep of the region of interest are acquired according to block 3. However, in later acquisition steps 2, only further diagnostic MRI data are acquired in blocks 5, while in blocks 3′ and 3″, which may use shorter total acquisition time as blocks 5, further PET raw data sets for the following acquisition steps 2 are acquired, wherein the acquisition times for the different patient table positions may not match those for the first PET raw data set of the first acquisition step 2 or patient table positions may even be skipped. For example, blocks 3′, 3″ may serve to increase recorded PET data, as, for example, described in U.S. Pat. No. 8,781,195 B2 already mentioned above. Also in such a case, the problem of patient motion occurring between the acquisition steps 2 exists when a full-count or at least multiple-count nuclear medical image data set, in this case PET data set, is to be determined.

Generally, in addition to the reconstruction of at least one nuclear medical image data set 6, in this case PET image data set 7, in a step S3, motion data describing the motion between acquisition steps 2 are determined from source data and used for motion correction in step S3. It is noted that motion data can also be used to adapt attenuation maps, if, like the case of FIG. 3, attenuation correction MRI data are only acquired during one attenuation step. As source data, PET data and/or additional modality data, here MRI data, may be used, wherein preferably both PET data and MRI data may be used to determine the motion data, since both modalities show different anatomical features.

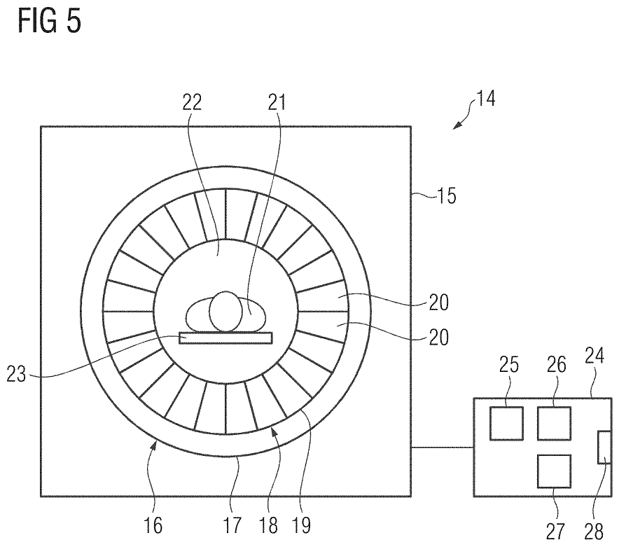

An exemplary flowchart for the determination of motion data 8 is shown in FIG. 4 for a pair of acquisition steps 2. Here, the nuclear medical raw data sets 9, in this case PET raw data sets 10, for each of the acquisition steps 2 are used in steps S4 to determine preliminary reconstructed images 11, in this case preliminary backprojected images, as source data 12. These preliminary reconstructed images 11 may be attenuation corrected (AC) or not attenuation corrected (NAC) using attenuation maps derived from the attenuation correction MRI data already discussed above. For example, Dixon techniques may be used to acquire attenuation correction MRI data in blocks 4, as principally known in the state of the art.

In a step S5, the preliminary reconstructed images 11 are registered using a registration algorithm to determine first motion data.

On the other hand, MRI data 13, preferably attenuation correction MRI data, are used as source data 12, in particular as input to a step S6, where the corresponding MRI images are registered to determine second motion data. The first and second motion data are then statistically combined to yield the final motion data 8.

It is noted that the use of attenuation correction MRI data is preferred, since, usually, Dixon techniques are employed yielding material distributions, which can easily and robustly be registered in a registration algorithm in step S6. However, it is also possible to use further MRI data, in particular diagnostic MRI data, or even to register MRI images acquired using different magnetic resonance sequences and/or protocols in step S6, for example if, like the case in FIG. 3, attenuation correction MRI data are not available for each acquisition step 2.

Motion data 8 can, of course, also be determined using other methods, for example optical flow algorithms and the like. Furthermore, further additional modalities may be used as a source of further motion data to be combined, for example cameras, in particular 3D cameras and/or terahertz cameras.

Returning to FIG. 1, in step S3, the motion data 8 are used to perform motion correction regarding the different acquisition steps 2. Here, multiple approaches are conceivable within the current invention, wherein, for example, in less preferred embodiments, a correction may be applied to already reconstructed nuclear medical image data sets 6. In preferred embodiments, however, motion correction is applied before or during reconstruction. If, for example, iterative reconstruction algorithms are used in step S2, for example motion sensitive OSEM reconstruction, the intermediate image may be warped before a forward projection and the correction terms resulting from comparison of the nuclear medical raw data and the forward projected data may be unwarped after backward projection. In this case, motion correction may be applied in sinogram space. However, in preferred embodiments, the motion data may also be applied in listmode space by reordering the lines of response of the events according to the motion information.

In the preferred case of dynamic nuclear medical imaging, a series of nuclear medical image data sets 6 for all the acquisition steps 2 is preferably determined, wherein the motion correction leads to pixel-wise correspondence between the nuclear medical image data sets 6. Furthermore, also in the case of dynamic nuclear medical imaging, a full-count nuclear medical image data set 6 may be determined by summing up the motion-corrected nuclear medical image data sets for all the acquisition steps 2. In cases like illustrated in FIG. 3, however, exactly one nuclear medical image data set 6, namely a full-count nuclear medical image data set 6, may be determined as the final result. In any case, the at least one nuclear medical image data set 6 is then provided for further processing, for example displaying on a display device of the imaging device, storing in a picture archiving system and/or further evaluation.

FIG. 5 shows an embodiment of an imaging device 14 according to the invention, in this case a PET-MR device 15 providing both modalities. In the schematical drawing of FIG. 5, the acquisition device 16 for the additional modality, in this case the MRI device 17, is only schematically indicated and provides, as known, a central bore, where the nuclear medical imaging device 18, in this case PET device 19, is located coaxially to the MRI device 17. The PET device 19 comprises multiple PET detection units 20 facing each other and arranged in pairs about the longitudinal direction (perpendicular to the image plane of FIG. 5). For example, the PET detection units 20 may comprise LSO crystals upstream of a photodiode array and/or an electrical amplifying circuit. Other concrete embodiments are also conceivable. An anatomical region of a patient 21 may be introduced into the field of view in the bore 22 of the imaging device 14 using a patient table 23, as already discussed above.

The operation of the imaging device 14 is controlled by a control device 24, whose functional structure is also partly indicated in FIG. 5. Generally said, the control device 24 is configured to perform a method according to one or more example embodiments of the present invention.

The control device 24 comprises an acquisition unit 25 to control the MRI device 17 and the PET device 19 to acquire respective data. Of course, separate acquisition units 25 for both modalities may also be provided. Hence, the at least one acquisition unit 25 is configured to perform step S1 of FIG. 1.

The control device 24 further comprises a reconstruction unit 26 for performing the reconstruction of step S2 and a motion correction unit 27 for performing the determination of motion data 8 and the motion correction according to step S3. Via an interface 28, the resulting at least one nuclear medical image data set 6 (PET image data set 7) may be provided, for example to a display device of the hybrid imaging device 14 (not shown).

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein and mentioned above, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’, ‘interface’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing system or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CDROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium, storage means or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been described in detail with reference to the preferred embodiment, the present invention is not limited by the disclosed examples from which the skilled person is able to derive other variations without departing from the scope of the invention.

1. A computer-implemented method for determining at least one nuclear medical image data set in nuclear medical imaging using an imaging device, the method comprising:
acquiring nuclear medical raw data sets of a region of interest of a patient in respective acquisition steps during a progression of a tracer in the region of interest;
reconstructing at least one nuclear medical image data set from the nuclear medical raw data sets for each acquisition step;
determining motion data describing a motion of the patient between acquisition steps from source data describing the motion in the region of interest; and
applying motion correction to at least one of the nuclear medical raw data sets or the at least one nuclear medical image data set based on the motion data.

2. The method of claim 1, wherein
the region of interest is larger than a field of view of the imaging device,
the acquiring includes at least one of
sweeping, during each acquisition step, the field of view a whole region of interest using at least one of multiple predefined positions of the field of view or a continuous movement of the field of view, or
at least one of (i) determining, for all acquisition steps, a series of nuclear medical image data sets or (ii) the region of interest comprises a whole body of the patient.

3. The method of claim 1, wherein the determining includes at least one of,
registering the source data relating to one of the acquisition steps to source data from another acquisition step to determine motion data between the acquisition steps, or
using optical flow algorithms to determine the motion data.

4. The method of claim 3, wherein the determining further includes at least one of,
registering neighboring acquisition steps in succession during the acquiring, from a first acquisition step or a last acquisition step, or
using preliminary reconstructed images from the nuclear medical raw data sets as source data to register.

5. The method of claim 1, wherein the applying includes,
applying the motion correction to a reconstruction result in an image space,
applying the motion correction in a raw data space during iterative reconstruction, or
the nuclear medical raw data sets are PET raw data sets, wherein lines of response are displaced according to the motion data for motion correction.

6. The method of claim 1, further comprising:
applying attenuation correction based on an attenuation map to the at least one nuclear medical image data set, and the applying the motion correction applies the motion correction to the attenuation map based on the motion data.

7. The method of claim 1, wherein the source data is at least partly acquired by an acquisition device of an additional modality different from the nuclear medical imaging during the acquisition steps, the acquisition device is registered to the imaging device.

8. The method of claim 7, wherein the acquisition device is at least one of,
at least one of a CT device or an MRI device integrated into the imaging device,
a camera,
a radar device, or
an ultrasound device.

9. The method of claim 7, further comprising:
applying attenuation correction based on an attenuation map, the attenuation map being determined from attenuation correction MRI data acquired by the acquisition device during at least one of the acquisition steps, the attenuation correction MRI data being used as at least a part of the source data.

10. The method of claim 9, wherein the acquiring includes,
acquiring acquisition correction MRI data in each acquisition step to determine a respective attenuation map for the acquisition step, or
acquiring acquisition correction MRI data only in one acquisition step, wherein, for the other acquisition steps, MRI data are acquired and at least partly used as source data.

11. The method of claim 1, wherein the source data is from different modalities.

12. The method of claim 1, wherein the determining the motion data determines motion data time-resolved within the acquisition steps.

13. An imaging device for determining at least one nuclear medical image data set in nuclear medical imaging, the imaging device comprising:
a control device including,
an acquisition unit configured to acquire nuclear medical raw data sets of a region of interest of a patient in respective acquisition steps during a progression of a tracer in the region of interest,
a reconstruction unit configured to reconstruct the at least one nuclear medical image data set from the nuclear medical raw data sets for each acquisition step, and
a motion correction unit configured to determine motion data describing a motion of the patient between acquisition steps from source data describing the motion in the region of interest and apply motion correction to at least one of the nuclear medical raw data sets or the at least one nuclear medical image data set based on the motion data.

14. A non-transitory electronically readable storage medium including instructions that, when executed by a control device of an imaging device, cause the imaging device to perform the method of claim 1.

15. A non-transitory electronically readable storage medium including instructions that, when executed by a control device of an imaging device, cause the imaging device to perform the method of claim 2.

16. The method of claim 5, wherein the source data is at least partly acquired by an acquisition device of an additional modality different from the nuclear medical imaging during the acquisition steps, the acquisition device is registered to the imaging device.

17. The method of claim 16, wherein the acquisition device is at least one of,
at least one of a CT device or an MRI device integrated into the imaging device,
a camera,
a radar device, or
an ultrasound device.

18. The method of claim 16, further comprising:
applying attenuation correction based on an attenuation map, the attenuation map being determined from attenuation correction MRI data acquired by the acquisition device during at least one of the acquisition steps, the attenuation correction MRI data being used as at least a part of the source data.

19. The method of claim 18, wherein the acquiring includes,
acquiring acquisition correction MRI data in each acquisition step to determine a respective attenuation map for the acquisition step, or
acquiring acquisition correction MRI data only in one acquisition step, wherein, for the other acquisition steps, MRI data are acquired and at least partly used as source data.

20. The method of claim 2, wherein the determining includes at least one of,
registering the source data relating to one of the acquisition steps to source data from another acquisition step to determine motion data between the acquisition steps, or
using optical flow algorithms to determine the motion data.
